(19) United States
(12) Patent Application Publication
(10) Pub. No.: US 2004/0248135 A1
(43) Pub. Date: Dec. 9, 2004

(54) Methods for detemining multiple effects of drugs that modulate function of transcription regulatory proteins

(76) Inventors: Daniel Notterman, Princeton, NJ (US); Thomas Shenk, Princeton, NJ (US)

Correspondence Address: Janet E Reed, Woodstock & Washburn, 46th Floor, One Liberty Place, Philadelphia, PA, 19103, US

(21) Appl. No.: 10/492,723

(22) PCT Filed: Oct. 18, 2002
(86) PCT No.: PCT/US02/33406

Related U.S. Application Data
(60) Provisional application No. 60/330,164, filed on Oct. 18, 2001.

(51) Int. Cl. C12Q 1/68

(52) U.S. Cl. 435/6

(57) ABSTRACT

Assays for determining the downstream effects of drugs that modulate the function of transcription regulatory proteins are disclosed. The assays comprise the steps of (a) providing cells that contain the transcription factor; (b) maintaining a control population and a test population of the cells under conditions that allow gene expression to occur in the cells, wherein the test population is exposed to the agent that modulates the activity of the transcription factor; (c) generating a gene expression profile for each of the control population and the test population of cells; and (d) comparing the gene expression profile from the control population of cells with the gene expression profile from the test population of cells; those differences being attributable to the effect of the agent that modulates the activity of the transcription factor. The assays are useful for classifying candidate transcription factor-modulating drugs on the basis of downstream gene expression effects, and thereafter correlating observed downstream effects with physiological markers of drug efficacy or side effects.

This application claims benefit of U.S. Provisional Application No. 60/330,164, filed Oct. 18, 2001, the entirety of which is incorporated by reference herein.

### FIELD OF THE INVENTION

This invention relates to the field of drug discovery and development. In particular the invention provides novel assays for determining the downstream effects of drugs that modulate the function of transcription regulatory proteins, for classifying such drugs on the basis of those effects, and thereafter correlating observed downstream effects with physiological markers of drug efficacy.

### BACKGROUND OF THE INVENTION

Various patent or scientific publications are referenced in this patent application to describe the state of the art to which the invention pertains. Each of these publications is incorporated by reference herein, in its entirety.

The human genome is estimated to contain more than 30,000 genes; other mammalian genomes may contain as many or more genes. Coordinated gene expression is essential for normal development and physiology. Aberrant gene expression is known to be a cause of numerous diseases or pathological conditions affecting humans and other mammals, one of the most notable being neoplastic disease, or cancer.

Gene expression is regulated at the transcriptional level by transcription regulatory proteins, or “transcription factors,” which interact with regulatory sequences of genes to modulate transcription of a variety of downstream genes. Examples of transcriptional regulators include tumor suppressor proteins such as p53, and nuclear receptor proteins such as the glucocorticoid receptor, estrogen receptor, thyroid hormone receptor and androgen receptor.

Each transcriptional regulator affects the expression of multiple downstream genes and the levels of their encoded mRNAs. Consequently, an alteration in the amount or activity of a transcription regulator will have a multiplicity of effects in the gene expression/signal transduction pathway(s) controlled by a given transcription factor; some of these effects may be therapeutically beneficial, while others may lead to disease states. Thus, while transcription regulatory proteins are tempting targets for therapeutic intervention, the multiplicity of downstream events that could be triggered by modulating the activity of a transcription regulatory protein presents a level of complexity in drug discovery that is challenging and difficult to overcome.

Screening assays for identifying compounds that modulate transcription factors have been developed. Customarily, such assays involve reconstituting an expression system by transforming cells with polynucleotides encoding the transcription factor along with a recombinant reporter gene containing a coding sequence for a detectable gene product and transcription control elements recognized and acted upon by the transcription factor. Transcription factor activity, in the presence or absence of a candidate drug, is measured by measuring the change in production of the detectable gene product. While such assays are very useful as an initial screen for transcription factor modulators, they do not provide information regarding the multiplicity of downstream events that would be affected by modulating the transcription factor. It would be an advance in the art to develop assays capable of providing information about these multiple downstream events and relating such information to the type or category of candidate drug being tested.

### SUMMARY OF THE INVENTION

This invention provides novel assays for determining the downstream effects of drugs that modulate the function of transcription regulatory proteins. Methods are provided for classifying such drugs on the basis of those effects, and thereafter correlating observed downstream effects with physiological markers of drug efficacy or side effects.

According to one aspect of the invention, an assay is provided for determining the effect of an agent that modulates activity of a transcription factor on expression of genes regulated by the transcription factor. The assay comprises following steps: (a) providing cells that contain the transcription factor; (b) maintaining a control population and a test population of the cells under conditions that allow gene expression to occur in the cells, wherein the test population is exposed to the agent that modulates the activity of the transcription factor; (c) generating a gene expression profile for each of the control population and the test population of cells; and (d) comparing the gene expression profile from the control population of cells with the gene expression profile from the test population of cells; differences between the gene expression profiles of the control population and the test population being attributable to the effect of the agent that modulates the activity of the transcription factor.

In another aspect, the invention features a method of classifying an agent that modulates activity of a transcription factor on the basis of the effect of the agent on expression of genes regulated by the transcription factor. This method comprises performing the steps outlined above on several transcription factor-modulating agents, then selecting one or more of the effects of each agent as a basis for classifying the agent.

In another aspect, the invention features a method for correlating physiological effects of (or a phenotypic feature associated with) a transcription factor-modulating agent with the effects of the agent on expression of genes regulated by the transcription factor. This type of correlation is used to advantage to select or eliminate candidate agents for further development, utilizing the downstream gene expression effect as a surrogate marker for the potential desirable or undesirable physiological effect.

In the foregoing assays, the gene expression profile is typically generated by a method comprising (a) providing an addressable population of single stranded nucleic acid molecules of a size sufficient to hybridize under pre-determined hybridization conditions with a complementary nucleic acid sequence of the same or greater size, wherein the addressable population comprises molecules corresponding to expressed genes in the target cell, wherein the target cell contains the transcription factor; (b) isolating mRNA from the target cells; and (c) contacting the addressable population of single stranded nucleic acid molecules with the isolated mRNA under conditions whereby the identity and amount of each mRNA produced by the target cells is quantifiably determinable by its hybridization to a complementary nucleic acid molecule in the addressable population of nucleic acid molecules. In certain embodiments, the addressable population of nucleic acid molecules comprises oligonucleotides corresponding to a cDNA library produced from the target cells or from an organism containing the target cells. In other embodiments, the addressable population of nucleic acid molecules comprises oligonucleotides corresponding to a set of genes known to be regulated by the transcription factor. In a preferred embodiment, the addressable population of nucleic acid molecules is spatially addressable by placement at a pre-determined location in an array on a solid support.

The assays of the invention may be practiced on any population of cells, including mammalian cells and, more specifically, human cells. The cells may be from normal or diseased tissue. Further, model systems comprising cells that naturally express, or have been engineered to express, certain transcription factors, or isotypes or mutants of a transcription factor, may be used. Any target transcription factor may be the subject of the assays of the invention. Nuclear receptors are particularly suitable targets, including but not limited to the estrogen receptor, glucocorticoid receptor, thyroid hormone receptor and androgen receptor.

Various features and advantages of the present invention will be understood by reference to the detailed description and examples that follow.

### DETAILED DESCRIPTION OF THE INVENTION

Transcriptional regulators modulate the expression of a variety of downstream genes. Many transcriptional regulators have been implicated in human disease. For instance, p53, the estrogen receptor and the androgen receptor are important in human cancers; and the glucocorticoid receptor is important in inflammation. Thus, transcriptional regulators represent important targets for the identification of new drugs. However, the complexity of screening for transcription regulator-modulating drugs with appropriate disease-treating activity is great, due to the fact that each transcriptional regulator affects the expression of multiple downstream genes; in some cases the expression of downstream genes is enhanced and in other cases it is reduced. Moreover, a transcription regulator may generate different downstream effects in a development- or tissue-specific fashion, lending even greater complexity to the assessment of transcription regulator-modulating agents. Still further adding to the complexity, many transcription regulators exist as multiple isotypes (the estrogen receptor and the glucocorticoid receptor being two examples), and more than one isotype may be present in a cell or tissue type. Moreover, mutants in some receptors have been associated with pathological conditions (e.g., one ER mutation results in estrogen hypersensitivity; various p53 mutations impair the tumor repressor functions of p53).

The methods of the present invention address these complexities through simultaneous monitoring of the expression at the mRNA level of a portion or all of the genes that are downstream of a specific transcriptional regulator. Thus, in the case of p53 for example, it is possible to monitor complete or partial reactivation of mutant p53 molecules and to identify drugs that block the regulation of p53 by other regulatory molecules such as hdm2, which is overexpressed in some tumors, blocking p53 function and contributing to oncogenesis. In the case of nuclear receptors, whose function can be altered in different ways by different drugs, the invention makes it possible to determine whether new drug candidates are related to previously studied drug candidates. This will provide a reliable means for classifying new compounds, as well as an indication of their likely side effects. Using the assays of the invention, drug discovery can be substantially facilitated because compounds can be grouped on the basis of downstream transcriptional effects and the groupings based on downstream mRNA levels can be correlated with known efficacious activities and known side effects, with changes in downstream mRNA levels serving as a surrogate marker for the physiological effects. This information will be very valuable because it will facilitate decisions as to which new drug candidates should be developed further and which new candidates should be discontinued (e.g., because they have similar effects on the surrogate markers as earlier candidates that proved unsatisfactory).

The sections that follow set forth embodiments for practicing the present invention. To the extent that specific reagents, molecules, cells, and the like are mentioned, they are merely illustrative and not intended to limit the invention.

The methods of the invention are used to confirm results obtained in a primary drug discovery screen, as well as to characterize the multiple downstream effects of such compounds. In certain embodiments of the invention, oligonucleotide or polynucleotide arrays are used to monitor the effect of a drug candidate on the expression of mRNAs that are controlled by a specific target protein. Drugs that alter the activity of a transcriptional regulatory protein would, of necessity, alter the expression of mRNAs whose expression is dependent on the regulator. For many regulatory proteins, different drugs will alter their activities in different ways, depending on where they bind to the regulator and possibly depending on the affinity of binding. Although some drugs will affect the expression of all mRNAs controlled by the regulatory protein, other drugs will alter some but not all downstream mRNAs. The changes in expression profile are monitored, e.g., by DNA array analysis. Thus, the assays of the invention can be used to categorize new drugs that specifically target transcriptional regulatory proteins in terms of their effects on expression of downstream genes and whether they are related in their activities to other known drugs or other modulatory compounds that have been only partially characterized. In preferred embodiments, the analysis is facilitated by the use of parallel processed microarray screening, to enable the simultaneous screening of multiple compounds on a single solid support containing multiple copies of the test array.

Thus, the particular utility of this drug discovery platform is that (1) it can monitor in detail changes in the activity of a transcription regulatory protein in response to a drug, and (2) it can provide a categorizing screen that will determine whether a drug that interacts with a transcription regulatory protein is novel or whether its activity suggests that it belongs to a previously identified class of drug. Consequently, the screen can determine whether a new drug candidate is likely to be novel in its activity or related to existing compounds in its activity. Importantly, the categorization can likely predict the side effects a new drug candidate might display, if the profile of its transcriptional effects is related to the profiles displayed by a group of previously studied compounds.

The assays of the invention are used to analyze and characterize candidate compounds which have been identified in a primary screen as modulators of the production or activity of a transcription regulatory protein. As used herein, the terms “transcription regulatory protein,” “transcription regulator” and “transcription factor” are interchangeable, all referring to a class of proteins whose function is to interact with transcriptional regulatory regions of genes and thus activate or repress (or increase or decrease) transcription of those genes.

Once a candidate compound has been identified, an assay of the invention is performed on a cell population or tissue known to contain the specified transcription regulatory protein. The cells or tissues are maintained under conditions that allow for normal cellular processes, specifically transcription/gene expression, to occur, in the presence or absence of the candidate compound. A gene expression profile is then generated, by isolating mRNA from the cells, then identifying and quantitating each mRNA produced by the cells. This is typically done through the use of an addressable population of nucleic acids that represent part or all of the expressed gene population of the particular cell type, or of the organism from which the cells are derived. More specifically, oligonucleotide arrays (i.e., “gene chips”) representing a particular population of expressed genes are used, as known in the art and described in greater detail below.

The gene expression profile of cells exposed to the candidate compound is compared to a profile from cells that have not been exposed to the compound, or to a profile from cells exposed to a known compound. The observed differences between the respective profiles contains information that can be used to classify or categorize the candidate compound, e.g., by one or more unique downstream gene expression differences or by the entire profile of differences, and can also yield valuable information connected to the type of downstream effects observed. For example, it may be observed that a particular class of compound modulates a selected transcription factor in a manner that affects the function of the transcription factor in one major pathway (e.g., cell cycle progression), but not another (e.g., inflammation). This type of information is expected to be extremely useful for the selection and development of transcription factor modulators that have predictable and desirable downstream effects within a cell.

The assays of the invention may be used to assess any compound identified as a candidate modulator of a specific transcription regulator, or class of transcription regulator. Many classes of transcription regulator have been identified to date, and many of these present attractive targets for therapeutic intervention (see, e.g., Emery et al. (2001) Therapeutic Modulation of Transcription Factor Activity, Trends in Pharmacol. Sci., 22: 233-240). One superclass of transcription regulators includes classes comprising a basic domain, for instance: basic leucine zipper (bZIP) transcription factors, including AP-1 and cAMP response element-binding protein (CREB); basic helix-loop-helix (bHLH) factors such as MyoD; and basic helix-loop-helix leucine zipper (bHLH-ZIP) factors such as the cell cycle-controlling factors Mch, Mad, Max and E2F and the sterol response element-binding proteins (SREBPs).

Another superclass of transcription regulators includes classes comprising zinc-coordinating DNA binding domains (zinc fingers). This important class includes SP1, Egr, Ga14, GATA, Krueppel and nuclear receptors (NR). NR represent particularly attractive targets for drug development. Examples of NR include glucocorticoid receptor, estrogen receptor, thyroid hormone receptor and androgen receptor.

Another superclass of transcription regulators comprises helix-turn-helix transcription factors, most notably the homeodomain transcription factors. Numerous members of this class have been linked to genetic disorders, reflecting the key role these regulators play in development and physiology. An example of a crucial homeobox transcription regulator is pancreatic duodenal homeobox 1 (PDX1) Moreover, certain homeodomain transcription regulators (e.g., Hoxc8) function as repressors in some instances. A high throughput screen for modulators of Hoxc8 has been described (Dirr et al., J. Bone Miner. Res. 15 Supp 1., S462, 2000). The assays of the present invention may be used to confirm and classify active agents identified in that initial screen.

Another superclass of transcription regulators contains classes containing β-scaffold factors with minor groove contacts. These classes include nuclear factors of activated T cells (NF-ATs), NF-κB, signal transducer and activator of transcription (STAT) and, notably, p53. Two modulators of members of this class include cyclosporin and FK506. The assays of the present invention can be utilized to classify and compare novel candidate drugs with these previously-characterized agents.

The assay is performed in a cell or tissue known to contain the transcription factor. Any such cell or tissue, from any organism, may be utilized, as would be appreciated by one of skill in the art. Specifically, mammalian cells are utilized. More specifically, human cells are utilized. In certain embodiments, additional information may be gained by performing the assay on cells or tissues in a diseased state; e.g., neoplastic cells or cells having metabolic anomalies associated with activity (or inactivity) of a particular transcription factor. When such cells are utilized, not only may gene expression profiles be evaluated, but also the phenotypic effect of the candidate compound on the cells may be evaluated. Furthermore, suitable cell types may be selected on the basis of which isoform(s) or mutant(s) of a transcription regulator they contain. For instance, breast cancers have been associated with an estrogen receptor (ER) mutant that renders cells hypersensitive to estrogen. Cultured cells of this type may be used to evaluate the downstream effects of candidate compounds that modulate the mutant ER.

In yet another embodiment, model systems may be utilized in which cells are engineered to express only one isotype or mutant of a transcription regulator. This may be accomplished by known means, e.g., by knocking out the production or activity of other isotypes in the cells, or by recombinantly adding a specified transcription factor isotype to a cell type that does not normally contain the transcription factor.

Once gene expression is allowed to occur in the selected cells, in the presence or absence of the candidate drug (or in a multiple parallel assay testing and comparing several candidate drugs), a gene expression profile for each sample of cells is generated. As mentioned, this is accomplished by isolating mRNA from the cells, and quantitatively identifying each mRNA represented in the sample by hybridization to addressable populations of sequence-determined oligonucleotides whose sequences correspond to expressed genes from that cell type, or from an organism from which the cell type was derived. The availability of cDNA libraries and other genomic information, along with the development of DNA microarrays and other technologies for assigning “addresses” to nucleic acid molecules of known sequences enable gene expression profiling to be accomplished in a multiple-parallel high-throughput manner.

In one embodiment, oligonucleotide arrays or microarrays disposed on a planar solid support (e.g., glass or plastic slide) are employed for generating gene expression profiles for the test samples. In a preferred embodiment, a multiple microarray system is utilized to facilitate parallel analysis of multiple test samples. Such systems are known in the art, and many are commercially available, (e.g., Affymetrix Inc., Santa Clara, Calif.; BD Biosciences/Clontech, Palo Alto, Calif.). Moreover, enabling microtechnology for performing high-throughput hybridization reactions is also available from several sources, (e.g., Caliper Technologies Corp., Mountain View, Calif.; Affymetrix, Inc., Santa Clara, Calif.).

In alternative embodiments, addressable populations of oligonucleotides that do not utilize the planar array platform may be used to generate gene expression profiles. For instance, an addressable oligonucleotide population may be generated by coating microbeads with individual oligonucleotide sequences. A unique detectable feature to is imparted to each bead (i.e., a unique dye), and that feature is correlated to the oligonucleotide coated upon the beads, thereby maling it identifiable (“addressable”), e.g., by flow cytometric means. Such “fluid arrays” are also commercially available (e.g., Luminex Corp., Austin, Tex., utilizing color-coded microbeads).

Yet other embodiments for generating a gene expression profile in a test sample may be utilized. These are known to persons skilled in the art, and many supporting technologies are commercially available.

Several approaches may be taken in the practice of the present invention. For instance, a candidate compound identified in a primary screen as a modulator of a transcription regulator may be further characterized by treating the selected cell type with the compound and generating a broad gene expression profile that measures most or all genes known to be expressed by that cell type. Oligonucleotide arrays of all known expressed genes or all known expressed genes combined with expressed sequence tags (ESTs) may be used for this purpose (e.g., Atlas cDNA arrays, Clontech; various GeneChips®, Affymetrix, Inc.). Alternatively, custom arrays to detect expression of specific downstream genes known to be regulated by a selected transcription factor may be utilized. As yet another alternative, a commercially available or custom array that represents genes associated with a disease or pathological condition (e.g., cancer) may be utilized. Other variations will be apparent to those of skill in the art.

The following examples are provided to describe the invention in greater detail. These examples are intended to illustrate, not to limit, the invention.

### EXAMPLE 1

### Comparison of Gene Expression Profiles in Cells Treated with a Known Estrogen Receptor Modulator and a New Candidate Estrogen Receptor Modulator

Tamoxifen is a widely used hormonal therapy for breast cancer. In breast tissue, tamoxifen exerts an anti-estrogenic effect, but in other tissue (e.g., cardiovascular system, bone, uterus), tamoxifen has estrogen-like effects. Novel candidate estrogen receptor (ER) modulators are compared with tamoxifen in their effect on expression of downstream genes.

Human breast cancer cells are cultured in the presence of either tamoxifen, the candidate compound, or carrier buffer alone. Messenger RNA is isolated from each cell sample population, and an expression profile for each sample is generated by hybridization to human cDNA expression arrays (e.g., Atlas arrays from Clontech or GeneChip arrays from Affymetrix). Results are recorded and analyzed using computer-facilitated devices. The gene expression profiles from each of the samples (tamoxifen-treated, candidate drug-treated, and control) are compared and differences among the samples are recorded and categorized. The candidate drug is classified by its similarities to and differences from tamoxifen.

In a subsequent experiment, sample cell populations as described above are analyzed by hybridizing the isolated mRNA to oligonucleotide arrays representing genes known to be regulated via the ER, or as part of a signal transduction pathway mediated by estrogen. These include genes involved in homeostasis and cell survival, as well as genes encoding stress response proteins and involved in apoptosis signal transduction, for instance, IL-7R-α, IGFBP 1 and 3, SHP2, Src, PI 3K and MAPK/Erk for a survival pathway, and RANTES, SOD-1, MLK 3, SAPK/JNK, c-Jun, AP-1, Cyclin B1 and CLK for an apoptosis pathway.

### EXAMPLE 2

### Confirmation of Candidate Modulators of the Glucocorticoid Receptor

The physiological response and sensitivity to glucocorticoids varies significantly among individuals and among various cell types within a single individual. Additionally, glucorcorticoid resistance or hypersensitivity respectively result in a number of pathological conditions. Candidate drugs that modulate the glucocorticoid receptor (GR) may be further screened to determine downstream gene expression events that may be desirable due to therapeutic utility, or that may need to be avoided due to pathogenic potential. Specifically, it is desirable to determine if a candidate drug up- or down-regulates signal transduction related to the inflammatory response, which is in part mediated through the glucocorticoid receptor.

GR-containing cells are cultured in the presence or absence of a candidate drug. Messenger RNA is isolated and an initial broad expression profile is generated using an array representing all expressed genes. Specific expression profiles are also generated by hybridizing the mRNA to arrays comprising genes encoding proteins that either (a) repress or (b) enhance the inflammatory response. The candidate compound is assessed on the basis of the broad expression profile of treated cells, and is further classified as an inflammation-promoting or inflammation-repressing drug on the basis of the specific expression profiles generated.

The present invention is not limited to the embodiments described and exemplified above, but is capable of variation and modification without departure from the scope of the appended claims.

We claim:

1. A method of determining the effect of an agent that modulates activity of a transcription factor on expression of genes regulated by the transcription factor, the method comprising:
a) providing cells that contain the transcription factor;
b) maintaining a control population and a test population of the cells under conditions that allow gene expression to occur in the cells, wherein the test population is exposed to the agent that modulates the activity of the transcription factor;
c) generating a gene expression profile for each of the control population and the test population of cells; and
d) comparing the gene expression profile from the control population of cells with the gene expression profile from the test population of cells; differences between the gene expression profiles of the control population and the test population being attributable to the effect of the agent that modulates the activity of the transcription factor.

2. The method of claim 1, wherein the gene expression profile is generated by a method comprising:
a) providing an addressable population of single stranded nucleic acid molecules of a size sufficient to hybridize under pre-determined hybridization conditions with a complementary nucleic acid sequence of the same or greater size, wherein the addressable population comprises molecules corresponding to expressed genes in the target cell, wherein the target cell contains the transcription factor
b) isolating mRNA from the target cells; and
c) contacting the addressable population of single stranded nucleic acid molecules with the isolated mRNA under conditions whereby the identity and amount of each mRNA produced by the target cells is quantifiably determinable by its hybridization to a complementary nucleic acid molecule in the addressable population of nucleic acid molecules.

3. The method of claim 2, wherein the addressable population of nucleic acid molecules comprises oligonucleotides corresponding to a cDNA library produced from the target cells or from an organism containing the target cells.

4. The method of claim 3, wherein the addressable population of nucleic acid molecules comprises oligonucleotides corresponding to a set of genes known to be regulated by the transcription factor.

5. The method of claim 2, wherein the addressable population of nucleic acid molecules is spatially addressable by placement at a pre-determined location in an array on a solid support.

6. The method of claim 1, wherein the cells are mammalian cells.

7. The method of claim 6, wherein the cells are human cells.

8. The method of claim 1, wherein the cells produce no more than a single form of the transcription factor.

9. The method of claim 1, wherein the transcription factor is a nuclear receptor.

10. The method of claim 9, wherein the transcription factor is selected from the group consisting of glucocorticoid receptor, estrogen receptor, thyroid hormone receptor and androgen receptor.

11. A method of classifying an agent that modulates activity of a transcription factor on the basis of the effect of the agent on expression of genes regulated by the transcription factor, the method comprising:
a) providing cells that contain the transcription factor;
b) maintaining a control population and a test population of the cells under conditions that allow gene expression to occur in the cells, wherein the test population is exposed to the agent that modulates the activity of the transcription factor;
c) generating a gene expression profile for each of the control population and the test population of cells;
d) comparing the gene expression profile from the control population of cells with the gene expression profile from the test population of cells; differences between the gene expression profiles of the control population and the test population being attributable to the effects of the agent that modulates the activity of the transcription factor on expression of genes regulated by the transcription; and
e) selecting one or more of the effects of the agent as a basis for classifying the agent.

12. The method of claim 11, wherein the gene expression profile is generated by a method comprising:
a) providing an addressable population of single stranded nucleic acid molecules of a size sufficient to hybridize under pre-determined hybridization conditions with a complementary nucleic acid sequence of the same or greater size, wherein the addressable population comprises molecules corresponding to expressed genes in the target cell, wherein the target cell contains the transcription factor
b) isolating mRNA from the target cells; and
c) contacting the addressable population of single stranded nucleic acid molecules with the isolated mRNA under conditions whereby the identity and amount of each mRNA produced by the target cells is quantifiably determinable by its hybridization to a complementary nucleic acid molecule in the addressable population of nucleic acid molecules.

13. The method of claim 12, wherein the addressable population of nucleic acid molecules comprises oligonucleotides corresponding to a cDNA library produced from the target cells or from an organism containing the target cells.

14. The method of claim 13, wherein the addressable population of nucleic acid molecules comprises oligonucleotides corresponding to a set of genes known to be regulated by the transcription factor.

15. The method of claim 14, wherein the addressable population of nucleic acid molecules is spatially addressable by placement at a pre-determined location in an array on a solid support.

16. The method of claim 11, wherein the cells are mammalian cells.

17. The method of claim 16, wherein the cells are human cells.

18. The method of claim 11, wherein the cells produce no more than a single form of the transcription factor.

19. The method of claim 11, wherein the transcription factor is a nuclear receptor.

20. The method of claim 19, wherein the transcription factor is selected from the group consisting of glucocorticoid receptor, estrogen receptor, thyroid hormone receptor and androgen receptor.

21. A method of correlating at least one physiological effect of an agent that modulates activity of a transcription factor with the effect of the agent on expression of genes regulated by the transcription factor, the method comprising:
a) providing cells that contain the transcription factor;
b) maintaining a control population and a test population of the cells under conditions that allow gene expression to occur in the cells, wherein the test population is exposed to the agent that modulates the activity of the transcription factor;
c) generating a gene expression profile for each of the control population and the test population of cells;
d) observing at least one physiological feature of each of the control population and the test population of cells;
e) comparing the phyisiological feature and the gene expression profile from the control population of cells with physiological feature and the gene expression profile from the test population of cells; differences between the gene expression profiles of the control population and the test population being attributable

24. The method of claim 23, wherein the addressable population of nucleic acid molecules comprises oligonucleotides corresponding to a set of genes known to be regulated by the transcription factor.

25. The method of claim 22, wherein the addressable population of nucleic acid molecules is spatially addressable by placement at a pre-determined location in an array on a solid support.

26. The method of claim 21, wherein the cells are mammalian cells.

27. The method of claim 26, wherein the cells are human cells.

28. The method of claim 21, wherein the cells produce no more than a single form of the transcription factor.

29. The method of claim 21, wherein the transcription factor is a nuclear receptor.

30. The method of claim 29, wherein the transcription factor is selected from the group consisting of glucocorticoid receptor, estrogen receptor, thyroid hormone receptor and androgen receptor.

31. The method of claim 21, performed upon a plurality of transcription-modulating agents, wherein the agents are classified on the basis of the correlation between their physiological effect and their effect on expression of genes regulated by the transcription factor.

32. The method of claim 31, further comprising the step of selecting and eliminating transcription modulating agents as candidates for drug development on the basis of the correlation or classification of the agents.
